(19) United States
(12) Patent Application Publication
(10) Pub. No.: US 2025/0352743 A1
(43) Pub. Date: Nov. 20, 2025

(54) SPIN CHAMBER FOR AN INHALER

(71) Applicant: AstraZeneca AB, Södertälje (SE)

(72) Inventors: Brian FOSTER, Raleigh, NC (US); Dan DEATON, Holly Springs, NC (US); Daniel COONEY, Raleigh, NC (US); James HANNON, Lafayette, CO (US); Eric RICHARDSON, Cave Creek, AZ (US); Peter HARLEY, Malmesbury (GB); Ben WADE, England (GB)

(21) Appl. No.: 18/666,615

(22) Filed: May 16, 2024

(51) Int. Cl. A61M 15/00

(52) U.S. Cl. CPC A61M 15/0008; A61M 15/0021; A61M 15/003; A61M 15/0086; A61M 2202/064

(57) ABSTRACT

A spin chamber for use in an inhaler includes a primary recess to receive air to mix with contents of a capsule. The primary recess has a curved wall. The spin chamber includes a secondary recess to hold a capsule. The secondary recess is located within a bottom surface of the primary recess. The spin chamber includes at least one curved inlet channel to allow air to travel therethrough. The curved inlet channel includes a tangential section and a funnel section. At least a portion of the tangential section is substantially tangential to the curved wall of the primary recess and is connected to an air inlet on an exterior surface of the spin chamber. The funnel section curves toward the primary recess and is connected to an entry point to allow air to enter therethrough into the primary recess.

### FIELD OF DISCLOSURE

The present disclosure relates to inhalers, and more specifically, to spin chambers for use in dry-powder inhalers.

### BACKGROUND

Inhalers are medical devices used to deliver a dose of medicament to a user by inhalation. There are numerous varieties of inhalers, but they all rely on the deliverance of the medicament into a user's lungs where the medicament may then be absorbed. Inhalers are used as a common treatment for asthma and chronic obstructive pulmonary disease (COPD), for example.

Dry powder inhalers are one such variety of inhaler. These deliver medicament to a user in the form of a dry powder, which is advantageous as this allows the medicament to reach further into the lungs than, for instance, metered dose or soft mist inhalers, thereby providing a greater therapeutic benefit to the user.

Existing dry powder inhalers, such as those described in EP 1,270,034 A2 and US 2007/295332 A1, may comprise spin chambers within which the medicament contained within a capsule can be released and then mixed with air travelling through the spin chamber.

Existing spin chambers may face issues such as powder migration from the spin chamber back into the inhaler, instead of out through the mouthpiece. This can affect the functions of other components of the inhaler. Existing spin chambers may also face issues with air flow due to their design. The surrounding geometry may cause disruption to the airflow, which may prevent the capsule from emptying its load and may result in a build up of powder in areas that do not receive an adequate airflow. As a result, a user may not receive a full dose of medication.

The present disclosure aims to solve these problems, among others.

### BRIEF SUMMARY

Aspects of the disclosure are as set out in the independent claims and additional features are set out in the dependent claims. Aspects of the disclosure may be provided in conjunction with each other and features of one aspect may be applied to other aspects.

An aspect of the disclosure provides a spin chamber for use in an inhaler, the spin chamber comprising: a primary recess for allowing air to mix with contents of a capsule, the primary recess having a curved wall for encouraging rotation of the capsule; a secondary recess for holding therein the capsule, the secondary recess located within a bottom surface of the primary recess; and at least one curved inlet channel through which air may travel, the at least one curved inlet channel defining a curved recess and comprising a tangential section and a funnel section, wherein: at least a portion of the tangential section is substantially tangential to the curved wall of the primary recess; the tangential section is connected at a first end to an air inlet on an exterior surface of the spin chamber through which air may enter the spin chamber and at a second end to a first end of the funnel section; and the funnel section curves in towards the primary recess and is connected at a second end to an entry point through which air may enter the primary recess, the funnel section being downstream from the tangential section; wherein the curved inlet channel is separated from the primary recess along a majority of its length by the curved wall of the primary recess.

Additionally, the spin chamber may have a longitudinal axis extending from a top of the spin chamber, down through the primary and secondary recesses, to a bottom of the spin chamber; the spin chamber may further comprise a top surface located at the top of the spin chamber with respect to the longitudinal axis; the primary recess may be proximate to the top of the spin chamber along the longitudinal axis, and the secondary recess may be proximate to the bottom of the spin chamber along the longitudinal axis; the bottom surface of the primary recess may face up towards the top of the inhaler with respect to the longitudinal axis; and the spin chamber may be configured so that in use air flows in from the air inlet, through the at least one curved inlet channel, through the primary recess and out through an outlet of the inhaler.

Additionally, the tangential section may comprise a first portion and a second portion, wherein: the first portion may extend from the first end of the tangential section to a point between the first end and the second end of the tangential section; the second portion may extend from the point between the first end and the second end of the tangential section to the second end of the tangential section; the second portion may be downstream from the first portion; the first portion may be widest near the air inlet; and the second portion may be of a substantially uniform width.

Additionally, the at least one curved inlet channel may comprise an inner wall and an outer wall, wherein: the inner wall may substantially follow an outline of the primary recess; the inner wall may extend along an entirety of the tangential section and along at least a portion of the funnel section; and the outer wall may be substantially straight in the first portion of the tangential section of the at least one curved inlet channel.

Additionally, the primary recess may be substantially cylindrical. This may help to encourage rotation of a capsule during inhalation.

Additionally, the secondary recess may be substantially obround-shaped with a length that is greater than its width, so as to receive a capsule horizontally relative to the longitudinal axis. This may help to ensure that a capsule can be perforated at both ends, thus resulting in a quicker and more efficient release of medication from the capsule. Additionally, the at least one curved inlet channel may have a length that is greater than a radius of the primary recess.

Additionally, the top surface of the spin chamber may be curved in a convex manner such that a depth along the longitudinal axis of the at least one curved inlet channel varies along its length. This curvature enables a drawer containing the spin chamber to be closed into the inhaler via a hinge mechanism, which results in a simplified user experience.

Additionally, the at least one curved inlet channel may comprise two curved inlet channels. This results in a greater air flow, which helps to lift the capsule and allow its contents to mix with the air.

Additionally, the two curved inlet channels may be on opposing sides of the primary recess. This means that air may interact with the capsule from both sides, which helps to lift the capsule and allow its contents to mix with the air.

Additionally, the tangential sections of each opposing curved inlet channel may be opposite each other across the primary recess and the funnel sections of each opposing curved inlet channel may be opposite each other across the primary recess.

Additionally, a first curved inlet channel of the two curved inlet channels may have a greater depth along the longitudinal axis in its tangential section than in its funnel section and wherein a second curved inlet channel of the two curved inlet channels has a greater depth in its funnel section than in its tangential section.

Additionally, a cross-sectional area of the air inlet of a first of the two curved inlet channels may be substantially equal to a cross-sectional area of the air inlet of a second of the two curved inlet channels. This may help to ensure a balanced air flow through both curved inlet channels, thus encouraging a stable cyclone to be generated in the spin chamber.

Additionally, the at least one curved inlet channel may be configured such that in use, air feeds into the primary recess, causing the capsule to be lifted out of the secondary recess and to spin in the primary recess. This helps to release the contents of the capsule quickly and efficiently, resulting in a greater likelihood of successful delivery.

Additionally, a bottom surface of the at least one curved inlet channel may be substantially level with the bottom surface of the primary recess with respect to the longitudinal axis.

Additionally, the primary recess may extend downwards from the top surface of the spin chamber along the longitudinal axis, and the at least one curved inlet channel may define a curved recess extending downwards from the top surface of the spin chamber.

Another aspect of the disclosure provides an inhaler comprising a spin chamber as described above.

Additionally, the inhaler may be configured to allow air to flow in from the air inlet, through the at least one curved inlet channel, through the primary recess and out through an outlet of the inhaler.

Additionally, the outlet of the inhaler may comprise a mouthpiece.

### BRIEF DESCRIPTION OF THE FIGURES

Embodiments of the disclosure will now be described, by way of example only, with reference to the accompanying drawings.

FIG. 1A shows a perspective view of an inhaler in accordance with the present disclosure.

FIG. 1B shows a perspective view of an inhaler with an open drawer in accordance with the present disclosure.

FIG. 2 shows an exploded view of an inhaler in accordance with the present disclosure.

FIG. 3A shows a top view of an inhaler with an open drawer in accordance with the present disclosure.

FIG. 3B shows a cross-sectional side view of an inhaler in accordance with the present disclosure.

FIG. 4A shows a top view of a spin chamber of an inhaler in accordance with the present disclosure.

FIG. 4B shows a top view of a curved inlet channel of the spin chamber of FIG. 4A in accordance with the present disclosure.

FIG. 5A shows a top view of the spin chamber of FIG. 4A, with a first line indicating a cross-section of the spin chamber along a first curved inlet channel and a second line indicating a cross-section of the spin chamber along a second curved inlet channel in accordance with the present disclosure.

FIG. 5B shows a cross-sectional view of the spin chamber of FIG. 5A along the first line in accordance with the present disclosure.

FIG. 5C shows a cross-sectional view of the spin chamber of FIG. 5A along the second line in accordance with the present disclosure.

FIG. 6 shows an internal view of air flow through an inhaler in accordance with the present disclosure.

FIG. 7 shows a cross-sectional side view of the chimney and the drawer being held together in accordance with the present disclosure.

### DETAILED DESCRIPTION

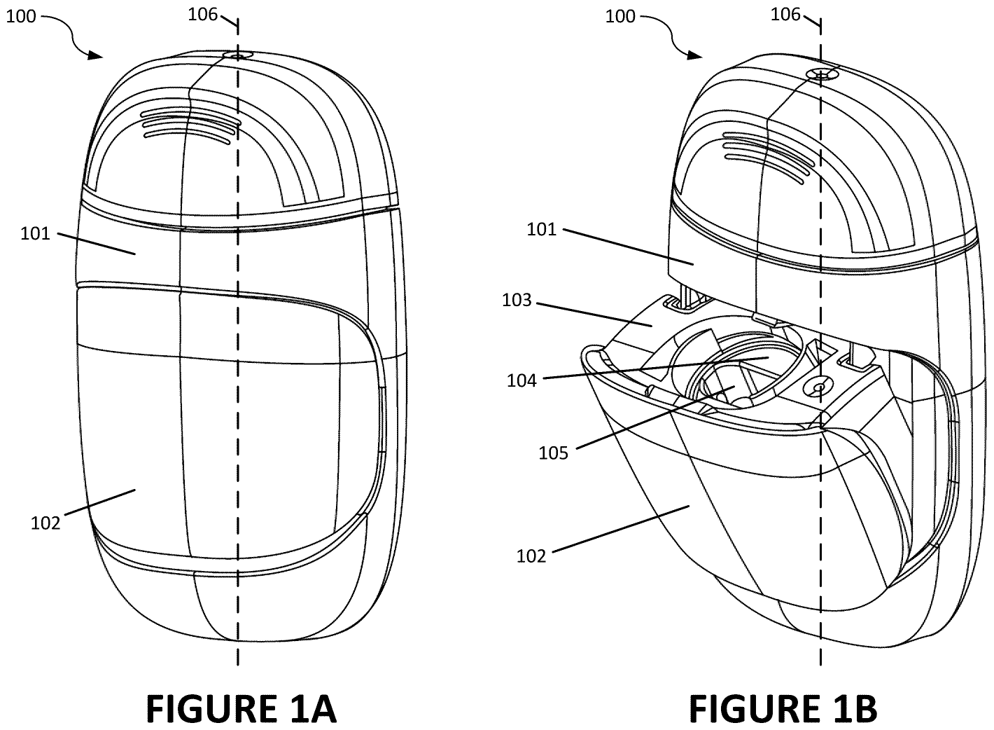

FIGS. 1A-B show perspective views of an inhaler 100. The inhaler 100 comprises a main body 101 and a drawer 102. The drawer 102 may be coupled to the main body by way of a hinge mechanism, which allows the drawer 102 to open out of and close into the main body 101 of the inhaler 100. This enables the drawer 102 to be accessed without having to remove it from the main body 101 entirely. The inhaler 100 may comprise a longitudinal axis 106, with the top of the inhaler 100 being positioned above the bottom of the inhaler 100 with respect to the longitudinal axis 106.

In FIG. 1A, the drawer 102 is shown as being in a closed position. In the closed position, a longitudinal axis of the drawer 102 may directly correspond to the longitudinal axis 106 of the inhaler 100. In FIG. 1B, the drawer 102 is shown as being in an open position, such that the components of the drawer 102 are visible. In the open position, the drawer 102 is angled outwards such that the longitudinal axis of the drawer is angled away from the longitudinal axis 106 of the inhaler 100.

The main body 101 is configured to act as a framework for the inhaler 100 and enclose the majority of the other components of the inhaler 100. The main body 101 may comprise polybutylene terephthalate (PBT) and at least a portion of the main body 101 may comprise wax-lubricated PBT. The main body 101 may comprise at least one air inlet to allow air to flow through the inhaler 100. The drawer 102 is configured to be opened out of and closed into the main body 101. More specifically, the spin chamber 103 of the drawer 102 is configured to receive a capsule and to allow the contents of the capsule to mix with air during inhalation. The contents of the capsule may comprise medicament in the form of a dry powder.

The drawer 102 comprises a spin chamber 103, which is located near the top of the drawer 102 with respect to the longitudinal axis 106. The spin chamber may comprise a primary recess 104 and a secondary recess 105. The primary recess 104 may extend downwards from the top surface of the spin chamber 103. The secondary recess 105 may be located within a bottom surface of the primary recess 104. As such, the secondary recess 105 can be considered as an extension of the primary recess 104. The primary recess 104 may be substantially cylindrical in shape, which may help to encourage rotation of a capsule during inhalation. The secondary recess 105 may be substantially obround in shape, with a length that is greater than its width. The primary recess 104 has a larger volume than the secondary recess 105.

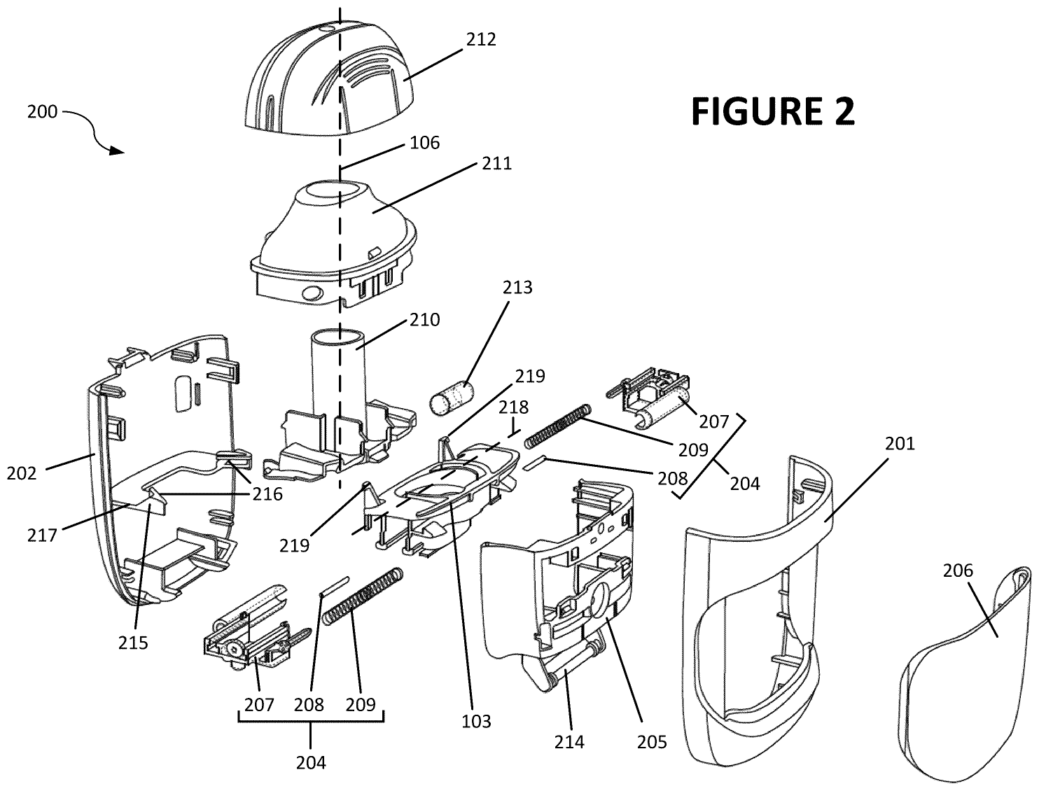

The secondary recess 105 is configured to receive the capsule. The obround shape of the secondary recess 105 enables the capsule to be received horizontally. This may help to ensure that a capsule can be perforated at both ends, thus resulting in a quicker and more efficient release of medication from the capsule. The process of perforation will be described with reference to FIG. 2. The primary recess 104 is configured to allow the contents of the capsule to mix with air during inhalation.

Use of the inhaler 100 begins with the insertion of a capsule into the drawer 102. The capsule is placed into the secondary recess 105 and the drawer 102 is closed into the main body 101. Closing the drawer 102 causes the capsule to be perforated, which will be described in greater detail with respect to FIG. 2. As the user inhales through the mouthpiece (not shown), the air flow through the spin chamber 103 causes the capsule to be lifted out of the secondary recess 105 into the primary recess 104, where it may spin such that its contents may mix with air flowing through the spin chamber 103. This mixture is then inhaled by the user. The drawer 102 may then be opened and the capsule removed.

The inhaler 100 and its use will be described in greater detail with respect to FIG. 2.

FIG. 2 shows an exploded view of an inhaler 200. The inhaler 200 may correspond to the inhaler 100 from FIGS. 1A-B and may therefore comprise a main body and a drawer, corresponding respectively to the main body 101 and the drawer 102 from FIGS. 1A-B.

More specifically, the main body of inhaler 200 may comprise a front casing 201 and a rear casing 202. The front casing 201 and rear casing 202 are connected to each other to provide a space within which other components of the inhaler 200 may be located. Each of the front casing 201 and rear casing 202 comprises an inner surface and an outer surface. When the front casing 201 and rear casing 202 are connected to each other, the two inner surfaces face inwards towards each other, while both outer surfaces face outwards. The front casing 201 and rear casing 202 both extend upwards along a longitudinal axis that may correspond to the longitudinal axis 106 from FIGS. 1A-B.

The front casing 201 comprises an aperture through which the drawer may move between an open position and a closed position. When the drawer is in the closed position, an outer surface of the drawer casing substantially fills the aperture of the front casing 201. When the drawer is in the open position, the components of the drawer are exposed, such that a capsule 213 may be inserted into or removed from the drawer. As described, the capsule 213 may contain medicament in the form of a dry powder.

The rear casing 202 may comprise at least one wedge 215, the at least one wedge 215 comprising an inner side 216 and being connected to a flexible arm 217. FIG. 2 shows an embodiment in which the rear casing 202 comprises two wedges 215, each comprising an inner side 216 and each attached to a separate flexible arm 217, but it is to be understood that fewer or more wedges 215 and flexible arms 217 are possible. In FIG. 2, the flexible arms 217 protrude outwards from the inner surface of the rear casing 202 along an axis that is substantially perpendicular to the longitudinal axis 106 of the rear casing 202.

The drawer of inhaler 200 may comprise a spin chamber 103, perforating means 204, a supporting framework 205 and a drawer casing 206. The spin chamber 103 may correspond to the spin chamber 103 from FIG. 1B and may comprise a transverse axis 218 that is substantially perpendicular to the longitudinal axis 106 of the inhaler and also substantially perpendicular to the axis along which the flexible arm 217 protrudes. The spin chamber 103, in addition to comprising a primary recess 104 and a secondary recess 105 for receiving a capsule 213, may also comprise at least one guide post 219. FIG. 2 shows an embodiment in which the spin chamber 103 comprises two guide posts 219, each located on opposing sides of the spin chamber 103 along the transverse axis 218. The guide posts 219 may extend upwards from a top surface of the spin chamber 103 substantially along the longitudinal axis 106.

The spin chamber 103 is coupled to the perforating means 204, which are positioned at a side of the spin chamber 103 along the transverse axis 218. The perforating means 204 are positioned so as to be able to move along the transverse axis 218 between a resting position and a perforating position. The perforating position is a position within the secondary recess 105 where the perforating means 204 may perforate the capsule 213. When in the resting position, the perforating means are further away from the centre of the spin chamber 103 than when in the perforating position. The spin chamber 103 may comprise rails to allow the perforating means 204 to slide along the transverse axis 218 between the resting position and the perforating position. The perforating means 204 may comprise grooves that interact with the rails of the spin chamber 103 to enable this movement. The spin chamber 103 may also comprise a T-rail (not shown) that helps to maintain alignment of the spin chamber 103 and the perforating means 103. The spin chamber 103 may further comprise perforating means retention clips (not shown) that prevent the perforating means 204 from moving outwards beyond their resting position along the transverse axis 218.

The spin chamber 103 and perforating means 204 may be coupled to the supporting framework 205, which holds the spin chamber 103 in a set position within the drawer. The supporting framework 205 also encloses the perforating means 204 within the drawer and may also help to prevent the perforating means 204 from moving outwards beyond their resting position along the transverse axis 218. A front side of the supporting framework 205 is attached to the drawer casing 206. The supporting framework may also comprise a hinge 214, which may be connected to the front casing 201 by way of a hook mechanism. The hook mechanism may have a substantially semi-circular cross section. The hinge 214 may also be connected to the rear casing 202. The presence of the hinge 214 may enable the drawer to be opened out of and closed into the main body while remaining attached to the main body. This enables the drawer to be accessed without having to remove it from the main body entirely.

The perforating means 204 may comprise a cam post 207, a needle 208 and a spring 209. The cam post 207 is coupled to a non-perforating end of the needle 208 and to a first end of the spring 209. The needle 208 and spring 209 both extend away from the cam post 207 along the transverse axis 218. The needle 208 may be encompassed by the spring 209, or it may be positioned away from the spring 209.

The second end of the spring 209 may be coupled to an inner portion of the perforating means 204, whereas the perforating end of the needle 208 is not directly connected to any other part of the inhaler. The spring 209 is in a rest state when the drawer is in the open position and when the drawer is in the closed position, but may be compressed as the drawer moves from the open position to the closed position, as will be described in greater detail.

FIG. 2 shows the perforating means 204 as comprising two sets of cam posts 207, needles 208 and springs 209, with each set located along the transverse axis 218 on opposing sides of the spin chamber 103, although the preceding paragraphs have so far described only one cam post 207, one needle 208 and one spring 209. It is to be understood that the inhaler may function with one cam post 207, one needle 208 and one spring 209, or with two cam posts 207, two needles 208 and two springs 209. The only requirements are that the perforating means comprises at least one cam post 207, at least one needle 208 and at least one spring 209. In an embodiment, the perforating means comprises two cam posts 207, two needles 208 and two springs 209, as shown in FIG. 2. In this embodiment, each of the two springs 209 may be coupled to the same inner portion of the perforating means at their respective second ends.

The two needles 208 may comprise a pair of opposing needles 208, each needle 208 coupled to a respective spring 209. The use of two opposing needles 208 may result in two perforations of the capsule 213. This decreases the time required for the contents of the capsule 213 to be removed from the capsule 213 through inhalation, since there will be two holes created in the capsule 213. The opposing needles 208 may be configured to perforate the capsule 213 at the same time. This helps to ensure an efficient and timely emptying of the capsule 213, since both holes will be created at the same time.

As described, the secondary recess 105 may be substantially obround-shaped. The needles 208 may be configured to enter opposing ends of the secondary recess 105 and subsequently perforate opposing ends of the capsule 213. This helps to ensure an efficient and timely emptying of the capsule, since this minimises the distance the contents of the capsule 213 will have to travel in order to exit the capsule 213.

As described, the perforating means 204 are configured to move along the transverse axis 218 between a resting position and a perforating position. More specifically, the cam post may be configured to transversely slide against the bias of the spring 209, which causes the spring 209 to compress. Since the needle 208 is attached to the cam post 207, the needle 208 may also be configured to transversely slide against the bias of the spring 209.

The movement of the drawer from an open position to a closed position may cause the perforating means 204 to move from the resting position to the perforating position.

The inhaler 200 may further comprise an inhalation chimney 210. The inhalation chimney 210 may comprise a hollow tube through which air and medicament may pass. The inhalation chimney 210 is positioned along the longitudinal axis 106 near the top of the inhaler, such that when the drawer is in the closed position, the inhalation chimney is directly above the spin chamber 103. The hollow tube extends along the longitudinal axis 106. The bottom of the hollow tube of the chimney 210 aligns with the primary recess 104 and secondary recess 105 of the spin chamber 103. When the drawer is in the closed position, the inhalation chimney 210 and the spin chamber 103 together define a space within which the contents of the capsule 213 may be spun as air travels through the inhaler 200.

The inhalation chimney 210 may also comprise at least one protruding rib along which the at least one guide post 219 of the spin chamber 103 may pass. The at least one protruding rib may extend outwards along the transverse axis 218. For example, there may be two protruding ribs on opposing sides of the inhalation chimney 210. The number of protruding ribs is the same as the number of guide posts 219.

The inhalation chimney 210 may also comprise at least one drawer retention clip (not shown). The at least one drawer retention clip may be situated near the bottom of the inhalation chimney 210 on the side that is closest to the rear casing 202. In an embodiment, the at least one drawer retention clip comprises two drawer retention clips on opposing sides of the inhalation chimney 210 with respect to the transverse axis 218.

The inhaler 200 may also comprise a mouthpiece 211. The mouthpiece 211 is positioned on top of the inhalation chimney 210 and comprises an aperture through which the inhalation chimney 210 may extend. The inhalation chimney 210 may move upwards along the longitudinal axis 106 such that a top surface of the inhalation chimney 210 is higher than a top surface of the mouthpiece 211 with respect to the longitudinal axis 106. The inhalation chimney 210 may move downwards along the longitudinal axis 106 such that the top surface of the inhalation chimney 210 is at the same level as the top surface of the mouthpiece 211 with respect to the longitudinal axis 106.

The mouthpiece 211 is attached to the front casing 201 and rear casing 202 of the inhaler 200.

The inhaler 200 may also comprise a cap 212. The cap 212 is positioned on top of the mouthpiece 211 and may cover the entire top surface of the mouthpiece 211. The cap 212 is attached to the mouthpiece 211 by way of a hinge mechanism that enables the cap 212 to either allow access to the mouthpiece 211 or to cover and prevent access to the mouthpiece 211.

With reference now to the function of the components of the inhaler 200, the front casing 201 and rear casing 202 are configured to act as the main body of the inhaler 200. The front casing 201 and rear casing 202 are joined to define an outer housing of the inhaler 200, within which other components may be enclosed.

The spin chamber 103, as has been described with reference to FIG. 1B, is configured to receive a capsule 213 and to allow air to mix with the contents of the capsule 213. More specifically, the secondary recess 105 of the spin chamber 103 is configured to receive the capsule 213. As air flows through the inhaler 200, the capsule 213 may be lifted out of the secondary recess 105 and into the primary recess 104, where the capsule 213 may spin around in order to allow its contents to mix with the air.

The perforating means 204 are configured to perforate the capsule 213, thus releasing the contents of the capsule 213 and allowing them to mix with air so that they may be inhaled by a user. More specifically, the perforating means 204 are configured to move inwards along the transverse axis 218 from a resting position to a perforating position as the drawer moves from an open position to a closed position. When at the perforating position, which occurs shortly before the drawer is in the closed position, the perforating means 204 are configured to perforate the capsule 213 and then move back from the perforating position to the resting position. When the drawer is in the closed position, the perforating means 204 are in the resting position. As the drawer moves from the closed position to the open position, the perforating means are configured to remain in the resting position.

The perforating means 204 are configured to interact with a portion of the main body of the inhaler 200 as the drawer moves between the open position and the closed position, which causes the perforating means to move away from their resting position towards their perforating position. More specifically, the perforating means are configured to interact with the wedge 215, which is attached to the flexible arm 217.

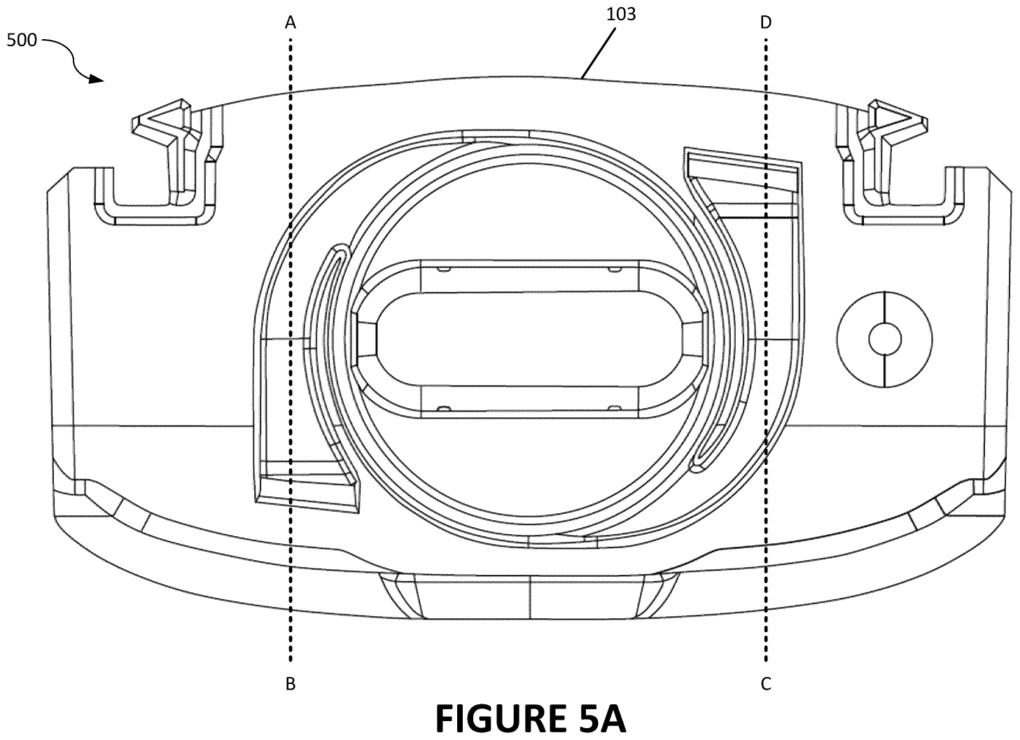

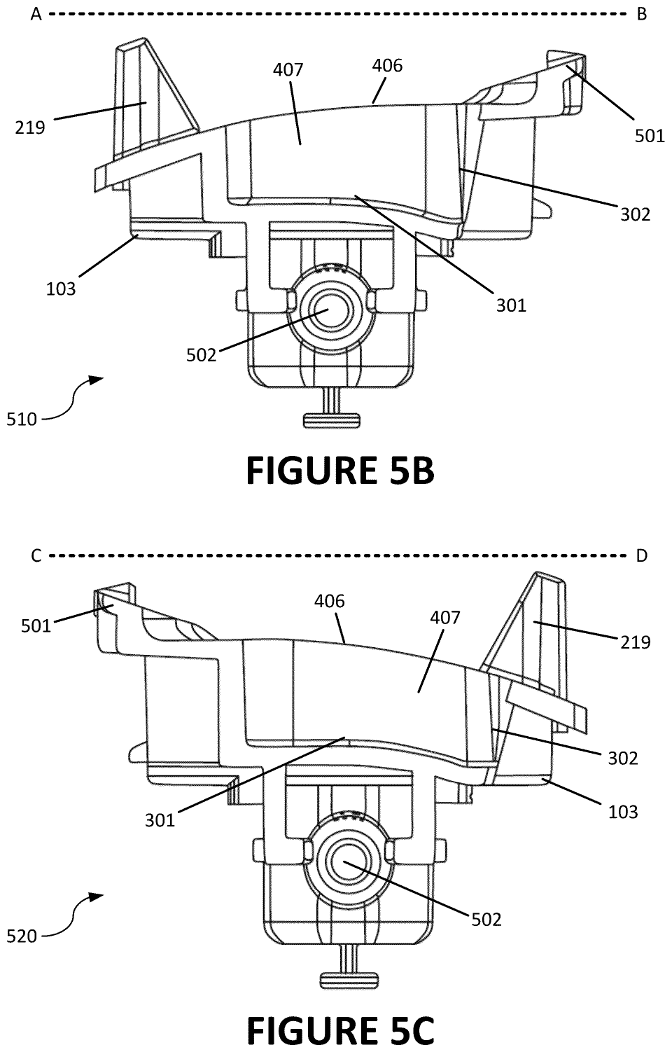

As the drawer moves into the main body of the inhaler 200 from the open position to the closed position, the cam post 207 of the perforating means 204 is configured to slide along the inner side 216 of the wedge 215. The angle of this inner side 216 causes the cam post 207 to be pushed inwards towards the centre of the spin chamber 103 along the transverse axis 218, against the biasing of the spring 209. This compresses the spring 209, which subsequently provides a resistive force. This helps to keep the other components of the perforating means 204 in the desired position. The needle 208, which is attached to the cam post 207, also moves inwards towards the centre of the spin chamber 103 and passes through a small aperture in the side of the spin chamber 103. Further details of this small aperture will be discussed with reference to FIGS. 5B-C. By the time the cam post 207 has reached the end of the inner side 216, the perforating means 204 have moved along the transverse axis 218 and have reached their perforating position. When in the perforating position, the needle 208 has extended through the small aperture in the side of the spin chamber 103 and into the secondary recess 105, where it may perforate the capsule 213.

This means that a capsule 213 can be perforated as the drawer is closed into the main body, rather than this being a separate step that must be initiated after the drawer has been closed. This makes use of the inhaler 200 easier and quicker for a user and also minimises the risk of a user failing to perforate a capsule (e.g. by not pressing a button hard enough), since the perforating means 204 must reach the perforating position in order for the drawer to successfully close.

Once the perforating position has been reached and the capsule 213 has been perforated, the perforating means 204 are configured to pass over the edge of the inner side 216 of the wedge 215 and in doing so return to the resting position. The compressed spring 209 decompresses and returns to its rest position. In doing so, the spring 209 pushes the needle 208 out of the secondary recess 105 such that the perforating means 204 can return to the resting position so that they are in the correct position for a subsequent opening of the drawer. At this point, the drawer is in the closed position. Beneficially, this means that a user does not have to manually reset the perforating means 204.

As the drawer is moved from a closed position to an open position, the perforating means 204 are configured to interact with the wedge 215, but in a different manner to the interaction that takes place when the drawer is being closed. As the drawer moves away from the closed position, the perforating means 204 are configured to travel over a top surface of the wedge 215. More specifically, the cam post 207 travels over the top surface of the wedge 215, which causes the flexible arm 217 to move downwards along the longitudinal axis 106 towards the bottom of the inhaler. As the cam post 207 travels over the wedge 215, the perforating means 204 remain in the resting position with respect to the transverse axis 218, meaning that the spring remains in the rest state. Once the cam post 207 has travelled over the top surface of the wedge 215, the wedge 215 moves back up to its normal resting position so that it is in the correct position for a subsequent closing of the drawer.

The inhalation chimney 210 is configured to move downwards with respect to the longitudinal axis 106 as the drawer moves from an open position to a closed position and is configured to move upwards with respect to the longitudinal axis 106 as the drawer moves from a closed position to an open position. More specifically, the guide posts 219 of the spin chamber are configured to interact with the inhalation chimney 210 as the drawer moves between the open and closed positions, which causes the inhalation chimney 210 to move upwards or downwards. When the drawer is in the closed position, the drawer retention clips are configured to hold the guide posts 219 in position, such that a force is required to move the guide posts 219 out of this position and open the drawer.

The mouthpiece 211 is configured to be inserted into a user's mouth during inhalation. The cap 212 is configured to cover the mouthpiece 211 when the inhaler 200 is not in use, thus preventing any foreign substances from entering the inhaler 200 through the mouthpiece 211.

In order to use the inhaler 200, a user may insert a capsule 213 into the secondary recess 105 of the spin chamber 103. The drawer must be in the open position for this to take place, since the spin chamber 103 cannot be accessed if the drawer is in the closed position. Once the capsule 213 is positioned within the secondary recess 105, the user may push the drawer inwards to move it from the open position towards the closed position. As the drawer moves towards the closed position, the perforating means 204 interact with the wedge 215, which causes them to slide along the inner side 216 of the wedge 215 and to move inwards along the transverse axis 218, as has been described.

The movement of the drawer causes the cam post 207 and the needle 208 to move inwards towards the centre of the spin chamber 103 along the transverse axis 218. As the cam post 207 approaches the edge of the wedge 215, the needle 208 perforates the capsule 213. The perforating means 204 then pass over the edge of the wedge 215 and return to the resting position.

The spin chamber 103 also interacts with the inhalation chimney 210 as the drawer moves from the open position towards the closed position. More specifically, the guide posts 219 of the spin chamber 103 slide along the protruding ribs of the inhalation chimney before travelling over sealing ramps of the inhalation chimney 210 as the drawer approaches the closed position. As the guide posts 219 travel over the sealing ramps, they cause the inhalation chimney 210 to be pulled downwards along the longitudinal axis 106, such that a bottom surface of the inhalation chimney 210 is brought closer to a top surface of the spin chamber 103. The two surfaces may be brought into contact, or a small gap may remain between them when the drawer is in the closed position. When the drawer is in the closed position, the inhalation chimney 210 has been pulled down such that a top surface of the inhalation chimney 210 is level with a top surface of the mouthpiece 211 with respect to the longitudinal axis 106. Drawer retention clips hold the guide posts 219 in position, such that the inhalation chimney 210 is held in position with respect to the spin chamber 103.

As discussed above, when the drawer is in the closed position, the perforating means 204 have perforated the capsule 213 and returned to the resting position and the inhalation chimney 210 has moved down towards the spin chamber 103. At this stage, the user may open the cap 212 to expose the mouthpiece 211. By placing the inhaler 200 in their mouth, tilting it and inhaling, an air flow may be generated through the inhaler 200. The air flow may lift the perforated capsule 213 out of the secondary recess 105 and into the primary recess 104 of the spin chamber 103, where it may cause the capsule 213 to spin and the contents of the capsule 213 to mix with the air. The resulting mixture of the contents of the capsule 213 and the air may then pass through the hollow tube of the inhalation chimney 210, through the aperture of the mouthpiece 211 and into the mouth of the user.

Upon successful inhalation, the drawer of the inhaler 200 may then be opened so that the capsule 213 may be removed. As the drawer is pulled outwards, the guide posts 219 push the drawer retention clips away and travel back over the sealing ramps. The guide posts 219 then interact with the protruding ribs of the inhalation chimney 210, which pushes the inhalation chimney 210 upwards with respect to the longitudinal axis 106.

At the same time, the perforating means 204 travel over the wedges 215. This movement pushes the wedges 215 downwards with respect to the longitudinal axis 106. The perforating means 204 therefore remain in the resting position as they travel over the wedges 215.

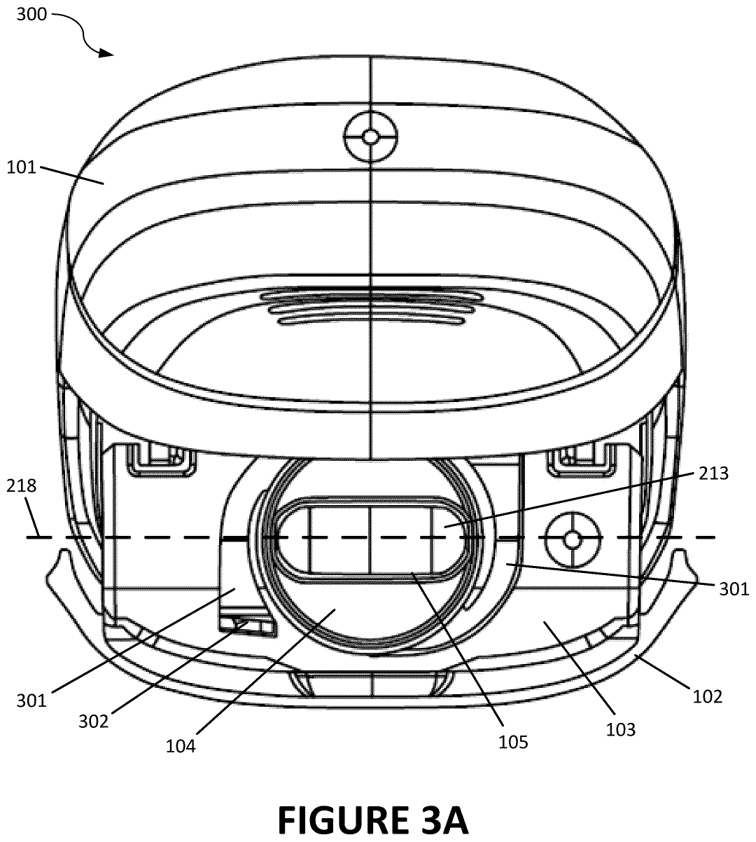

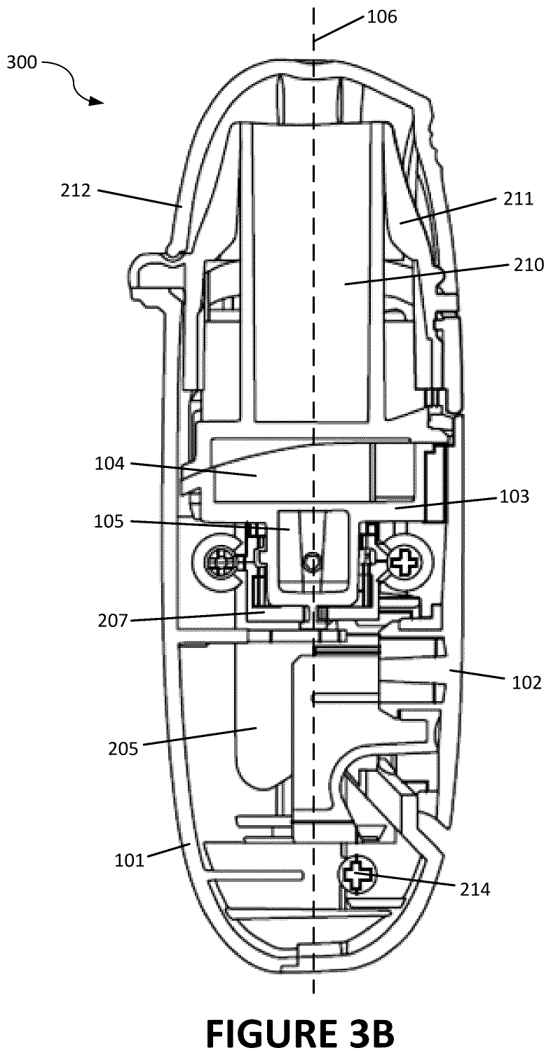

FIG. 3A shows a top view of an inhaler 300 with an open drawer in accordance with the present disclosure. FIG. 3B shows a cross-sectional side view of an inhaler 300 in accordance with the present disclosure. The inhaler 300 may be the same as the inhaler 100 from FIG. 1 and the inhaler 200 from FIG. 2.

Referring firstly to FIG. 3A, the spin chamber 103 is shown in greater detail. As has been discussed, the spin chamber 103 comprises a primary recess 104 and a secondary recess 105. The secondary recess 105 is configured to receive the capsule 213.

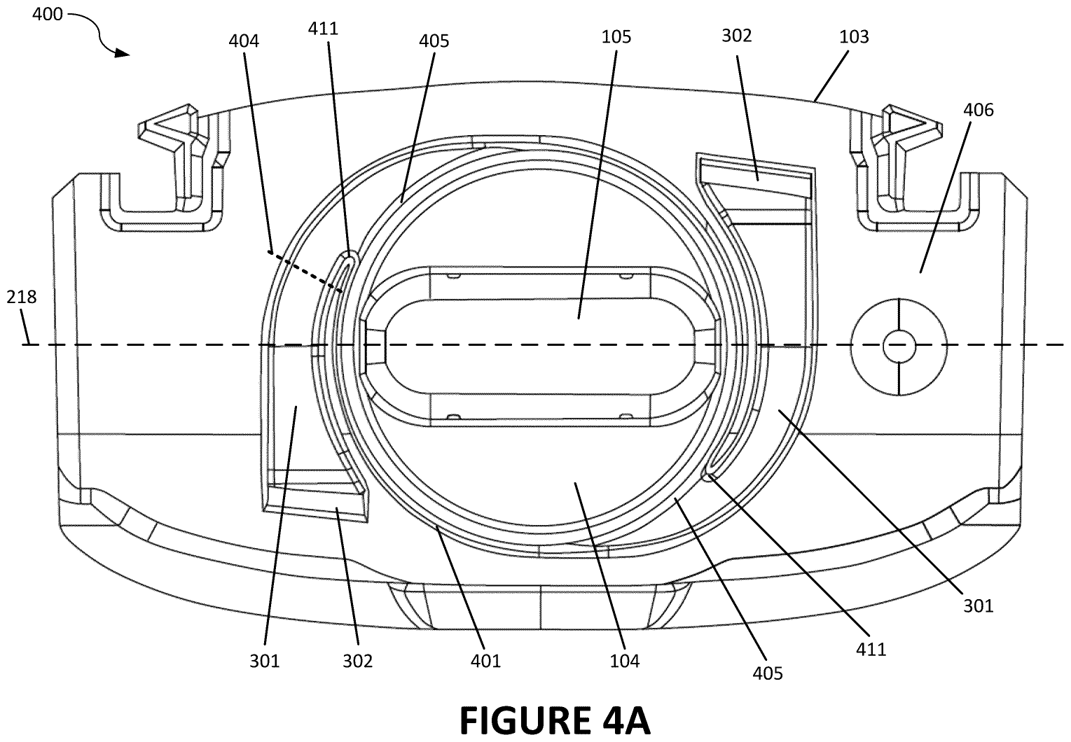

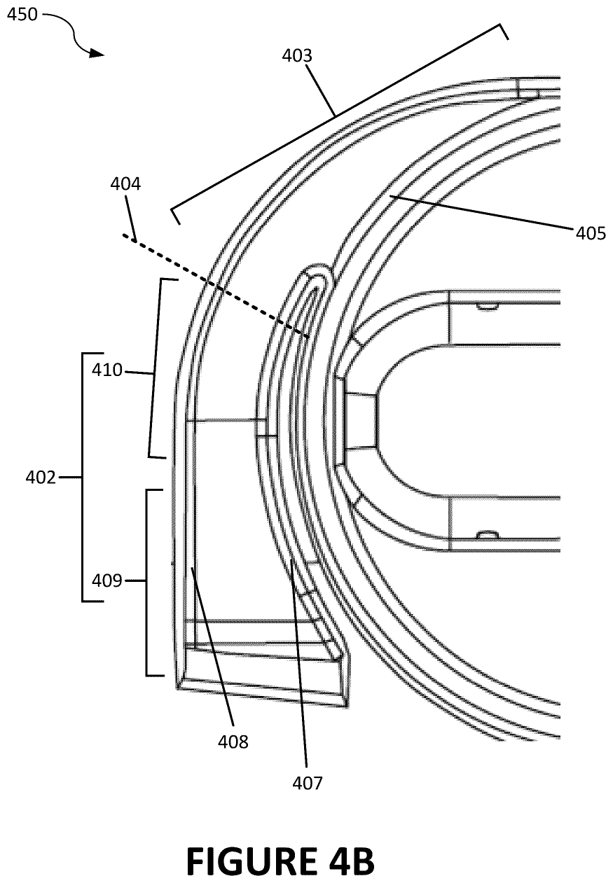

The spin chamber 103 may also comprise at least one curved channel 301, through which air may travel from at least one air inlet 302 into the primary recess 104. The air may then mix with the contents of the capsule 213 during inhalation. FIG. 3A shows an embodiment in which the spin chamber 103 comprises two curved channels 301. The curved channels 301 may be separated from the primary recess 104 along a majority of their length by a curved wall. Features of the curved channels 301 will be described in greater detail with reference to FIGS. 4A-B and 5A-C.

Referring now to FIG. 3B, the internal structure of the inhaler 300 when in the closed position is shown. As has been described with reference to FIG. 2, the components of the drawer 102 are enclosed within the main body 101 when the drawer 102 is in the closed position. The inhalation chimney 210 may extend through the mouthpiece 211, which itself is covered by the cap 212. The spin chamber 103 is positioned at the top of the drawer 102, such that when the drawer 102 is in the closed position, the spin chamber 103 is directly underneath the inhalation chimney 210 with respect to the longitudinal axis 106. The spin chamber 103 is coupled to the supporting framework 205, which is attached to the main body 101 by way of a hinge mechanism 214. The cam post 207 is coupled to the side of the spin chamber 103 and is able to move inwards with respect to the transverse axis, but not upwards or downwards with respect to the longitudinal axis 106.

Referring now to both FIGS. 3A and 3B, as has been described, when the drawer 102 is closed into the main body 101 from an open position towards a closed position, the perforating means are configured to interact with a portion of the main body 101, which causes the perforating means to move from a resting position to a perforating position, where the perforating means may perforate a capsule held in the secondary recess 105. As the drawer 102 continues to move towards the closed position, the perforating means return to the resting position and the inhalation chimney 210 is pulled downwards with respect to the longitudinal axis 106, such that a chamber may be defined. As will be described with reference to FIG. 8, this chamber may comprise the primary recess 104, the secondary recess 105 and a volume defined by the inhalation chimney 210. During inhalation, air may travel through the air inlets 302, along the curved channels 301 and into the primary recess 104, into which the capsule 213 has been lifted and the contents of the capsule have begun to empty. The air may then mix with the contents of the capsule 213 as the capsule 213 is spun around by the air.

FIG. 4A shows a top view 400 of a spin chamber 103 of an inhaler, such as the inhaler 100. The spin chamber 103 may be the same spin chamber from FIG. 1. FIG. 4B also shows a top view 450 of the spin chamber 103, but zoomed in further. Both of FIGS. 4A-B therefore show top views of the same spin chamber 103 and will therefore be discussed in tandem.

As with the inhaler 100, the spin chamber 103 may have a longitudinal axis 106 extending from a top of the spin chamber 103 to the bottom of the spin chamber 103. The spin chamber may comprise a top surface 406 that faces upwards with respect to the longitudinal axis 106. The spin chamber 103 may also comprise a primary recess 104 and a secondary recess 105. The primary recess 104 may extend downwards with respect to the longitudinal axis 106 from the top surface 406 of the spin chamber 103. The secondary recess 105 may be located within a bottom surface of the primary recess 104 and may also extend downwards with respect to the longitudinal axis 106. As such, the secondary recess 105 can be considered as an extension of the primary recess 104.

The primary recess 104 may be substantially cylindrical in shape and the secondary recess 105 may be substantially obround in shape. The primary recess 104 has a larger volume than the secondary recess 105. The primary recess 104 may be located substantially near the centre of the top surface 406 with respect to the transverse axis 218.

The primary recess 104 may comprise a curved wall 401 that extends around a majority of the primary recess in a substantially circular configuration. The curved wall 401 may substantially enclose the primary recess 104.

The spin chamber 103 also comprises at least one curved inlet channel 301, through which air may travel from at least one air inlet 302 on an exterior of the spin chamber 103 into the primary recess 104. The at least one curved inlet channel 301 may be separated from the primary recess 104 along a majority of its length by the curved wall 401 of the primary recess 104. The at least one curved inlet channel 301 may define a curved recess extending downwards from the top surface 406 of the spin chamber. The bottom surface of the curved inlet channel 301 may be substantially flat along a majority of its length. In this way, a bottom surface of the at least one curved inlet channel 301 may be substantially level with the bottom surface of the primary recess 104.

The at least one curved inlet channel 301 may comprise a tangential section 402 and a funnel section 403. The two sections may be separated by a boundary 404. The boundary 404 may be a point along the channel 301, as shown in FIG. 4A, but may instead be a small region of the channel 301 rather than a specific point. The tangential section 402 may be connected at a first end to the air inlet 302 on the exterior surface of the spin chamber 103 and at a second end to the boundary 404. The funnel section 403 may be connected at a first end to the boundary 404 and at a second end to an entry point 405 through which air may enter the primary recess 104 from the channel 301. The entry point 405 may be a wide opening in the curved wall 401 and may be located near to an end of the secondary recess 105, meaning that, in use, the entry point 405 is located near an end of a capsule. In the embodiment shown in FIG. 4A, the boundary 404 is a point, meaning that the second end of the tangential section 402 is connected to the first end of the funnel section 403. The two sections are arranged such that the funnel section 403 is downstream from the tangential section 402.

At least a portion of the tangential section 402 may be substantially tangential to the curved wall 401 of the primary recess 104. The tangential section 402 may comprise a first section 409 and a second section 410. The first section 409 of the tangential section 402 may extend from the first end of the tangential section 402 to a point between the first end of the tangential section 402 and the second end of the tangential section 402. This point may be located approximately half way along a length of the tangential section 402, although it may be located closer to the second end of the tangential section 402 than the first end of the tangential section 402. Alternatively, this point may be located closer to the first end of the tangential section 402 than the second end of the tangential section 402.

The second portion 410 of the tangential section 402 may extend from the point between the first and second ends of the tangential section 402 to the second end of the tangential section 402. The two portions are arranged such that the second portion 410 is downstream from the first portion 409.

The first portion 409 may be widest near the air inlet 302, which, as described above, is located at the first end of the channel 301. A width of the first portion 409 may then decrease with distance downstream, which equates to distance along the channel 301 from the air inlet 302 towards the entry point 405. The second portion 410 may have a substantially uniform width.

The at least one curved inlet channel 301 may comprise an inner wall 407 and an outer wall 408. Both walls may face inwards with respect to the channel 301 and as such may directly face one another. The inner wall 407 may be located closer to the centre of the spin chamber 103 than the outer wall 408. The inner wall 407 may be curved and may substantially follow an outline of the primary recess 104. Since the shape of the primary recess 104 is defined by the curved wall 401, the curvature of the inner wall 407 may substantially match the curvature of the curved wall 401. The inner wall may extend from the air inlet 302, along the entirety of the tangential section 402 and along at least a portion of the funnel section 403 until it reaches the entry point 405. At the entry point 405, the inner wall 407 combines with the curved wall 401 at an edge 411.

The outer wall 408 may be substantially straight in the first portion 409 of the tangential section 402 of the channel 301. In the second portion 410 of the tangential section 402, the outer wall 408 may begin to curve inwards towards the primary recess 104, such that it substantially matches a curvature of the inner wall 407 and the curved wall 401 of the primary recess 104. In the funnel section 403 of the at least one curved inlet channel 301, the outer wall 408 may continue to curve in towards the primary recess 104. The outer wall 408 therefore extends along an entirety of the channel 301. At the entry point 405, the outer wall 408 is substantially tangential to the primary recess 104 and may combine with the curved wall 401 of the primary recess 104. Although the inner wall 407 joins with the curved wall 401 at an edge 411, the tangentiality of the outer wall 408 with the primary recess 104 means that the outer wall 408 joins with the curved wall 401 in a substantially seamless manner. A length of the at least one curved inlet channel 301 may be greater than a radius of the primary recess 104.

As mentioned above, the spin chamber 103 comprises at least one curved inlet channel 301. FIGS. 4A-B show an embodiment in which the at least one curved inlet channel 301 comprises two curved inlet channels 301. This results in a greater air flow through the inhaler, which helps to lift the capsule and allow its contents to mix with the air. The two curved inlet channels 301 may be on opposing sides of the primary recess 104. This means that air may interact with the capsule from both sides, which helps to lift the capsule and allow its contents to mix with the air. A first of the two channels 301 may extend from a first air inlet 302 on a first exterior surface of the spin chamber 103 to a first entry point 405, while a second of the two channels 301 may extend from a second air inlet 302 on a second exterior surface of the spin chamber 103 to a second entry point 405. The second exterior surface may be on an opposite side of the spin chamber 103 to the first exterior surface and the second entry point 405 may be directly opposite the first entry point 405 across the primary recess 104.

The positions of the two curved inlet channels 301 with respect to each other can also be explained geometrically. Taking the top view 400 of the spin chamber 103 to be a planar, two-dimensional grid, and taking the centre of the primary recess 104 to be the centre of the grid, the second of the two channels 301 can be considered to represent substantially a 180-degree rotation of the first of the two channels 301 about the centre of the grid. As such, the tangential sections 402 of each opposing curved inlet channel 301 may be opposite each other across the primary recess 104 and the funnel sections 403 of each opposing curved inlet channel 301 may be opposite each other across the primary recess 104.

The two channels 301 may have different height profiles, which may arise due the curvature of the top surface 406. This will be described in greater detail with respect to FIGS. 5A-C. The first air inlet 302 may have a different height to the second air inlet 302 as a result of the different height profiles of the two channels 301. In order to ensure a balanced air flow through the inhaler, the first air inlet 302 may also have a different width to the second air inlet 302. More specifically, the relative heights and widths of the two air inlets 302 may be chosen such that a cross-sectional area of the first air inlet 302 is substantially equal to a cross-sectional area of the second air inlet 302. This may help to ensure a balanced air flow through both curved inlet channels 301, thus encouraging a stable cyclone to be generated in the spin chamber 103.

As described earlier with reference to FIGS. 1A-B, 2 and 3A-B, the primary recess 104 is configured to allow air to mix with the contents of a capsule, such as the capsule 213 from FIG. 2. The secondary recess 105 is configured to hold therein the capsule and keep it in a position for perforation, such that its contents may be released. The arrangement of the primary recess 104 and the secondary recess 105 allows for the capsule to be lifted out of the secondary recess 105 and up into the primary recess 104 during inhalation. The curved wall 401 of the primary recess 104, which defines the shape of the primary recess 104, encourages rotation of the capsule during inhalation, thus helping to release its contents and allow them to mix with air.

The at least one curved inlet channel 301 is configured to allow air to travel into the primary recess 104, where it may mix with the contents of the capsule during inhalation, as described above.

The inner wall 407 helps to direct the airflow towards the primary recess 104. The extension of the inner wall 407 into the funnel section 403 helps to reduce the migration of any powder (that may have been released from the capsule) from the primary recess 104 back through the channel 301 and into other parts of the inhaler. Such migration is undesirable because it prevents a user from receiving a full dose of medicament and may also cause blockages in other parts of the inhaler. The arrangement of the channel 301, and in particular the positions and lengths of its inner wall 407 and outer wall 408, therefore help to eliminate powder spillage through the inlet 302.

The positions and dimensions of the inner wall 407 and the outer wall 408 also define the position and dimensions of the entry point 405. This arrangement means that flow stagnation areas in the channel 301 is eliminated and also helps to ensure that air entering the channel 301 is less susceptible to disruption by surrounding geometry. The location of the entry point 405 also helps to focus air towards the ends of a capsule located in the secondary recess 105, thus helping to lift the capsule and ensure that its contents can mix with the air.

In use, a user of an inhaler comprising the spin chamber 103, such as inhaler 100, will insert a capsule into the secondary recess 105 and then close the drawer of the inhaler. This will perforate the capsule, as has been described with reference to earlier Figures. The user may then inhale through a mouthpiece of the inhaler, causing air to enter the spin chamber 103 through the inlets 302. The air will then travel through the curved inlet channels 301, firstly through the first portion 409 of the tangential section 402, then through the second portion 410 of the tangential section 402, and then through the funnel section 403, before entering the primary recess 104 at the entry point 405. The curved wall 401 of the primary recess causes a vortex to form as the air moves around through the primary recess 104, which lifts the capsule out of the secondary recess 105 and into the primary recess 104. When in the primary recess 104, the capsule is spun around, helping to release its contents, which can then mix with the air. The resulting mixture is then inhaled by the user through the mouthpiece, which functions as an outlet.

FIG. 5A shows a top view 500 of a spin chamber 103 that is identical to the top view 400 from FIG. 4A. The spin chamber 103 may be the same spin chamber 103 from the previous Figures. The top view 500 includes a first line A-B extending along a first of the two curved inlet channels 301 and a second line C-D extending along a second of the two curved inlet channels 301.

FIG. 5B shows a cross-sectional view 510 of the spin chamber 103 along the first line A-B. FIG. 5C shows a cross-sectional view 520 of the spin chamber 103 along the second line C-D. The first line A-B extends through and along a first of the two curved inlet channels 301 and the second line extends through and along a second of the two curved inlet channels 301.

With reference to both FIGS. 5B-C, the cross-sectional view 510 shows the spin chamber 103 comprising at least one curved inlet channel 301. Here, an embodiment shows the spin chamber 103 as comprising two curved inlet channels 301. A first of these channels 301 can be seen in FIG. 5B and a second of these channels 301 can be seen in FIG. 5C. As has been described with reference to previous Figures, each curved inlet channel 301 is connected at a first end to an air inlet 302, which is located on an exterior surface of the spin chamber 103. Each curved inlet channel 301 comprises an inner wall 407 that extends along the channel 301. The curved inlet channels 301 may comprise bottom surfaces that are substantially flat along a majority of their respective lengths.

The spin chamber 103 also comprises at least one guide post 219. FIGS. 5A-C show an embodiment in which the spin chamber comprises two guide posts 219, both located on the same side of the spin chamber 103 but at different ends with respect to the transverse axis 218. The guide posts 219 may extend upwards from a top surface 406 of the spin chamber 103 with respect to the longitudinal axis 106.

The spin chamber 103 may also comprise a pair of small apertures 502, each located on opposing sides of the secondary recess of the spin chamber 103. The spin chamber 103 may also comprise a front bridge 501 that is located on the top surface 406 of the spin chamber 103. The front bridge 501 may be a locking mechanism that extends upwards with respect to the longitudinal axis 106. The front bridge 501 may be positioned along a side of the top surface 406.

The top surface 406 may be curved in a convex manner such that, when the spin chamber 103 is closed inside the inhaler, one side extends higher up the longitudinal axis 106 than the other side. More specifically, the side that is closest to the front casing extends higher up the longitudinal axis 106 than the side that is closest to the rear casing. This can be seen in FIGS. 5B-C, where the side comprising the front bridge 501 extends higher up the longitudinal axis 106 than the side comprising the guide posts 219. The curvature of the top surface 406 enables a drawer containing the spin chamber 103 to be closed into the inhaler via a hinge mechanism, which results in a simplified user experience.

Since the top surface 406 is curved, but the bottom surfaces of the curved inlet channels 301 are substantially flat and level, the depths of the curved inlet channels 301 with respect to the longitudinal axis vary along their lengths. Since the two curved inlet channels 301 have air inlets 302 on opposing sides of the spin chamber 103, the two curved inlet channels 301 have different depth profiles along their lengths. Here, the depth should be interpreted as the distance from the top surface 406 of the spin chamber 103 to the bottom surface of the curved inlet channel 301 with respect to the longitudinal axis 106.

Referring specifically to FIG. 5B, the first of the two curved inlet channels 301 comprises an air inlet 302 located on a side of the spin chamber 103 that extends higher up the longitudinal axis 106 than its opposing side. The first curved inlet channel 301 therefore has its greatest depth at the air inlet 302. Proceeding inwards along the channel 301 (from B to A), the bottom surface of the first curved inlet channel 301 may curve upwards slightly before flattening out. This, combined with the downwards curvature of the top surface 406, provides a distinct depth profile in which the depth of the first curved inlet channel 301 decreases with distance along the channel 301. The first curved inlet channel 301 therefore has a greater depth along the longitudinal axis 106 in its tangential 402 section than in its funnel section 403.

Referring now specifically to FIG. 5C, the second of the two curved inlet channels 301 comprises an air inlet 302 located on a side of the spin chamber 103 that extends lower down the longitudinal axis 106 than its opposing side. The second curved inlet channel 301 therefore has its smallest depth at the air inlet 302. Proceeding inwards along the channel 301 (from D to C), the bottom surface of the first curved inlet channel 301 may curve upwards slightly before flattening out. This, combined with the upwards curvature of the top surface 406, provides a distinct depth profile in which the depth of the first curved inlet channel 301 slightly increases with distance along the channel 301. This depth profile is therefore different from the depth profile of the first curved inlet channel 301. The entry point of each channel 301 may therefore have a different depth with respect to the longitudinal axis 106, despite having a substantially identical width. The second curved inlet channel 301 therefore has a greater depth in its funnel section 403 than in its tangential section 402.

The functions of most of the components in FIGS. 5A-C have already been described with reference to previous Figures. The front bridge 501 is configured to lock onto at least a portion of the inhalation chimney when the inhaler is closed, thus helping to keep the components of the inhaler aligned and sealed during inhalation. The small apertures are configured to allow the needles to pass through them into the secondary recess and perforate the capsule as the drawer is closed into the main body, as has already been described with reference to FIG. 2.

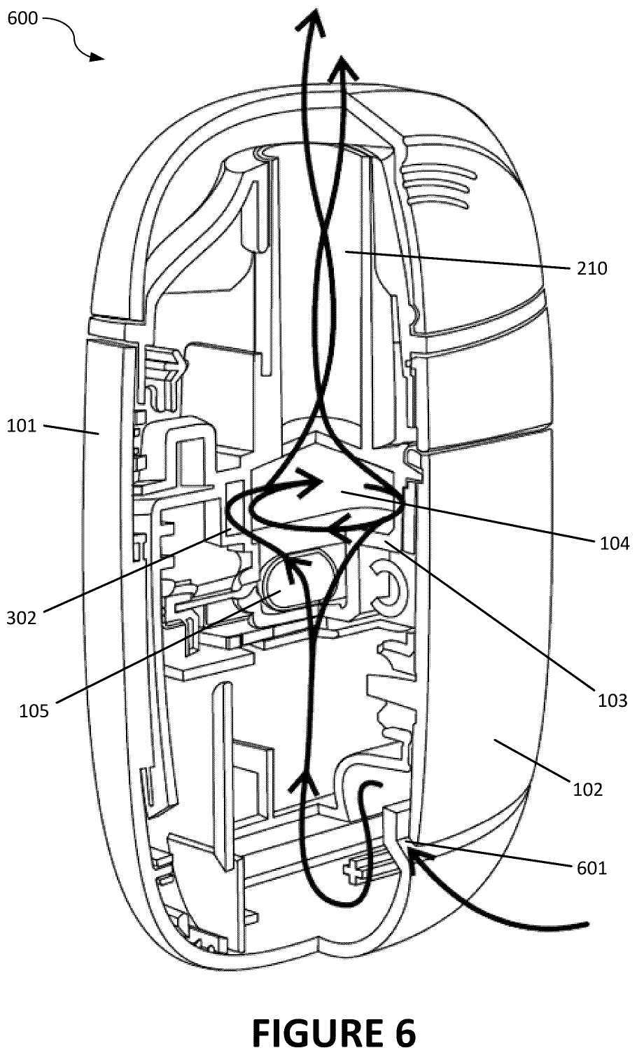

FIG. 6 shows an internal view 600 of air flow through a closed inhaler during inhalation, such as inhaler 100 from FIG. 1. The internal view is semi cross-sectional, as it shows the inhaler with a segment removed to visualise the internal components.

The inhaler may comprise an air inlet 601 of the inhaler through which air may enter the inhaler. FIG. 6 shows this air inlet 601 as being positioned near the bottom of the inhaler with respect to the longitudinal axis 106, specifically underneath the bottom of the drawer 102, but it should be understood that the air inlet 601 may be located at a number of other positions within the inhaler. It should also be understood that multiple air inlets 601 are possible.

The air then travels upwards through the inside of the inhaler as the user inhales. The air enters the spin chamber 103 through the air inlets 302 of the spin chamber 103, where it may pass along the curved inlet channels and into the primary recess 104. The movement of the air through the primary recess 104 causes a perforated capsule to be lifted out of the secondary recess 105 into the primary recess 104, where the air may spin the capsule around. The contents of the capsule can then mix with the air, and this mixture may then travel upwards through the inhalation chimney 210 and into the mouth of the user.

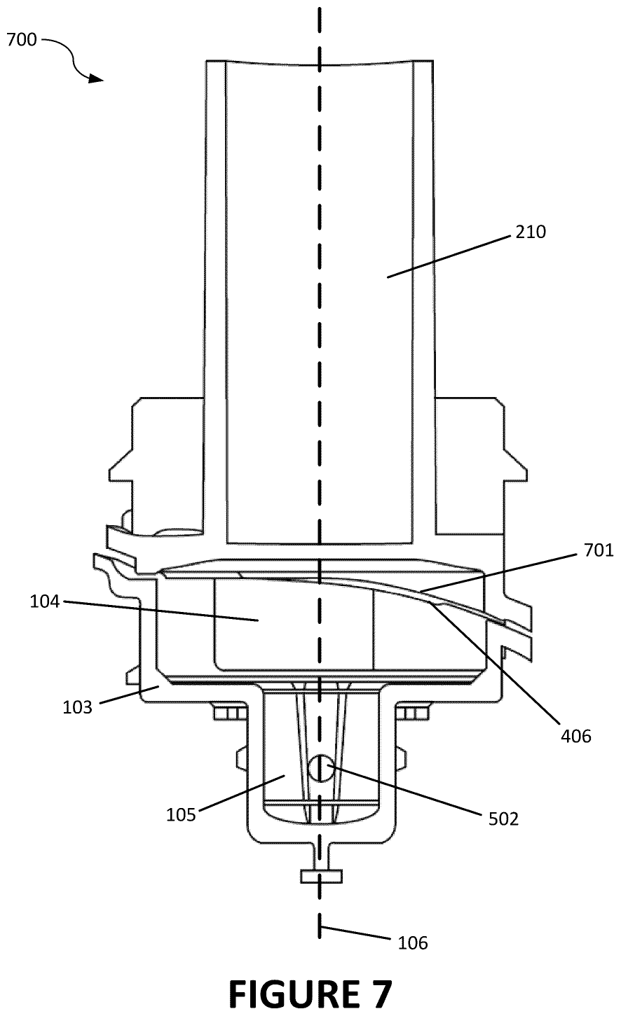

FIG. 7 shows a cross-sectional side view 700 of the inhalation chimney 210 and the spin chamber 103 being held together in accordance with the present disclosure.

When the drawer is in the closed position, the inhalation chimney 210 is positioned directly above the spin chamber 103 with respect to the longitudinal axis 106. The spin chamber 103 may comprise a top surface 406 facing upwards with respect to the longitudinal axis 106. The top surface 406 may also be described as a top surface of the drawer, since the spin chamber 103 is located at the top of the drawer. The top surface 406 of the spin chamber 103 may be curved in a convex manner, as can be seen in FIG. 7.

The inhalation chimney 210 may comprise a bottom surface 701 facing downwards with respect to the longitudinal axis 106. The bottom surface 701 may be curved in concave manner corresponding to the curve of the top surface 406 of the spin chamber 103.

The top surface 406 and the bottom surface 701 are configured to be held together during inhalation, in order to define a chamber within which air can mix with the contents of a capsule inserted into the inhaler. This chamber may comprise the primary recess 104, the secondary recess 105 and a volume defined by the inhalation chimney 210. The curves of the two surfaces correspond to one another so that the spin chamber 103 and the inhalation chimney 210 may enclose the chamber.

When the drawer is in the closed position, the inhalation chimney 210 has been pulled downwards with respect to the longitudinal axis 106, as has been described. There may still be a small gap present between the top surface 406 and the bottom surface 701. As a user inhales through the mouthpiece of the inhaler, the negative pressure caused by the inhalation may cause the spin chamber 103 to move upwards slightly such that the top surface 406 and bottom surface 701 are in direct contact with each other. In this way, a seal may be formed between the two surfaces.

Various embodiments of the present disclosure include one or more of the following items:

1. A spin chamber for use in an inhaler, the spin chamber comprising: a primary recess configured to receive air to mix with contents of a capsule, the primary recess having a curved wall configured to allow rotation of the capsule; a secondary recess configured to hold the capsule, the secondary recess located within a bottom surface of the primary recess; and at least one curved inlet channel configured to allow air to travel therethrough, the at least one curved inlet channel defining a curved recess and comprising a tangential section and a funnel section, wherein: at least a portion of the tangential section is substantially tangential to the curved wall of the primary recess; the tangential section is connected at a first end to an air inlet on an exterior surface of the spin chamber and at a second end to a first end of the funnel section, wherein the air inlet is configured to allow air to enter therethrough into the spin chamber; and the funnel section curves toward the primary recess and is connected at a second end to an entry point configured to allow air to enter therethrough into the primary recess, wherein the funnel section is downstream from the tangential section; wherein the curved inlet channel is separated from the primary recess along a majority of its length by the curved wall of the primary recess.

2. The spin chamber of item 1, wherein: the spin chamber has a longitudinal axis extending from a top of the spin chamber, down through the primary and secondary recesses, to a bottom of the spin chamber; the spin chamber comprises a top surface located at the top of the spin chamber with respect to the longitudinal axis; the primary recess is proximate to the top of the spin chamber along the longitudinal axis, and the secondary recess is proximate to the bottom of the spin chamber along the longitudinal axis; the bottom surface of the primary recess faces the top of the inhaler with respect to the longitudinal axis; and the spin chamber is configured so that in use air flows in from the air inlet, through the at least one curved inlet channel, through the primary recess and out through an outlet of the inhaler.

3. The spin chamber of item 2, wherein the tangential section comprises a first portion and a second portion, wherein: the first portion extends from the first end of the tangential section to a point between the first end and the second end of the tangential section; the second portion extends from the point between the first end and the second end of the tangential section to the second end of the tangential section; the second portion is downstream from the first portion; the first portion is widest near the air inlet; and the second portion is of a substantially uniform width.

4. The spin chamber of item 3, wherein the at least one curved inlet channel comprises an inner wall and an outer wall, wherein: the inner wall substantially follows an outline of the primary recess; the inner wall extends along an entirety of the tangential section and along at least a portion of the funnel section; and the outer wall is substantially straight in the first portion of the tangential section of the at least one curved inlet channel.

5. The spin chamber of any of items 2 to 4, wherein the primary recess is substantially cylindrical.

6. The spin chamber of any of items 2 to 5, wherein the secondary recess is substantially obround-shaped with a length that is greater than its width such that the secondary recess is configured to receive a capsule horizontally relative to the longitudinal axis.

7. The spin chamber of any of items 2 to 6, wherein the at least one curved inlet channel has a length that is greater than a radius of the primary recess.

8. The spin chamber of any of items 2 to 7, wherein the top surface of the spin chamber is curved in a convex manner such that a depth along the longitudinal axis of the at least one curved inlet channel varies along its length.

9. The spin chamber of any of items 2 to 8, wherein the at least one curved inlet channel comprises two curved inlet channels.

10. The spin chamber of item 9, wherein the two curved inlet channels are disposed on opposing sides of the primary recess.

11. The spin chamber of item 10, wherein the tangential sections of each opposing curved inlet channel are opposite each other across the primary recess, and wherein the funnel sections of each opposing curved inlet channel are opposite each other across the primary recess

12. The spin chamber of any of items 9 to 11, wherein a first curved inlet channel of the two curved inlet channels has a greater depth along the longitudinal axis in its tangential section than in its funnel section and wherein a second curved inlet channel of the two curved inlet channels has a greater depth in its funnel section than in its tangential section.

13. The spin chamber of any of items 9 to 12, wherein a cross-sectional area of the air inlet of a first of the two curved inlet channels is substantially equal to a cross-sectional area of the air inlet of a second of the two curved inlet channels.

14. The spin chamber of any of items 2 to 13, wherein the at least one curved inlet channel is configured such that in use air feeds into the primary recess, thereby causing the capsule to be lifted out of the secondary recess and to spin in the primary recess.

15. The spin chamber of any of items 2 to 14, wherein a bottom surface of the at least one curved inlet channel is substantially level with the bottom surface of the primary recess with respect to the longitudinal axis.

16. The spin chamber of any of items 2 to 15, wherein the primary recess extends downwards from the top surface of the spin chamber along the longitudinal axis, and the at least one curved inlet channel defines a curved recess extending downwards from the top surface of the spin chamber.

17. An inhaler comprising the spin chamber of any of items 1 to 16.

18. The inhaler of item 17, wherein the inhaler is configured to allow air to flow in from the air inlet, through the at least one curved inlet channel, through the primary recess and out through an outlet of the inhaler.

19. The inhaler of item 18, wherein the outlet of the inhaler comprises a mouthpiece.

It will be appreciated from the discussion above that the embodiments shown in the Figures are merely exemplary, and include features which may be generalized, removed or replaced as described herein and as set out in the claims.

Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the disclosure, which is defined in the accompanying claims.

Where ranges are recited herein these are to be understood as disclosures of the limits of said range and any intermediate values between the two limits.

With reference to the drawings in general, it will be appreciated that schematic functional block diagrams are used to indicate functionality of systems and apparatus described herein. It will be appreciated however that the functionality need not be divided in this way and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

Method embodiments may be implemented using the apparatus described herein.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the disclosure, which is defined in the accompanying claims.

These claims are to be interpreted with due regard for equivalents.

1. A spin chamber for use in an inhaler, the spin chamber comprising:
a primary recess configured to receive air to mix with contents of a capsule, the primary recess having a curved wall configured to allow rotation of the capsule;
a secondary recess configured to hold the capsule, the secondary recess located within a bottom surface of the primary recess; and
at least one curved inlet channel configured to allow air to travel therethrough, the at least one curved inlet channel defining a curved recess and comprising a tangential section and a funnel section, wherein:
at least a portion of the tangential section is substantially tangential to the curved wall of the primary recess;
the tangential section is connected at a first end to an air inlet on an exterior surface of the spin chamber and at a second end to a first end of the funnel section, wherein the air inlet is configured to allow air to enter therethrough into the spin chamber; and
the funnel section curves toward the primary recess and is connected at a second end to an entry point configured to allow air to enter therethrough into the primary recess, wherein the funnel section is downstream from the tangential section;
wherein the curved inlet channel is separated from the primary recess along a majority of its length by the curved wall of the primary recess.

2. The spin chamber of claim 1, wherein:
the spin chamber has a longitudinal axis extending from a top of the spin chamber, down through the primary and secondary recesses, to a bottom of the spin chamber;
the spin chamber comprises a top surface located at the top of the spin chamber with respect to the longitudinal axis;
the primary recess is proximate to the top of the spin chamber along the longitudinal axis, and the secondary recess is proximate to the bottom of the spin chamber along the longitudinal axis;
the bottom surface of the primary recess faces the top of the inhaler with respect to the longitudinal axis; and
the spin chamber is configured so that in use air flows in from the air inlet, through the at least one curved inlet channel, through the primary recess and out through an outlet of the inhaler.

3. The spin chamber of claim 2, wherein the tangential section comprises a first portion and a second portion, wherein:
the first portion extends from the first end of the tangential section to a point between the first end and the second end of the tangential section;
the second portion extends from the point between the first end and the second end of the tangential section to the second end of the tangential section;
the second portion is downstream from the first portion;
the first portion is widest near the air inlet; and
the second portion is of a substantially uniform width.

4. The spin chamber of claim 3, wherein the at least one curved inlet channel comprises an inner wall and an outer wall, wherein:
the inner wall substantially follows an outline of the primary recess;
the inner wall extends along an entirety of the tangential section and along at least a portion of the funnel section; and
the outer wall is substantially straight in the first portion of the tangential section of the at least one curved inlet channel.

5. The spin chamber of claim 2, wherein the primary recess is substantially cylindrical.

6. The spin chamber of claim 2, wherein the secondary recess is substantially obround-shaped with a length that is greater than its width such that the secondary recess is configured to receive a capsule horizontally relative to the longitudinal axis.

7. The spin chamber of claim 2, wherein the at least one curved inlet channel has a length that is greater than a radius of the primary recess.

8. The spin chamber of claim 2, wherein the top surface of the spin chamber is curved in a convex manner such that a depth along the longitudinal axis of the at least one curved inlet channel varies along its length.

9. The spin chamber of claim 2, wherein the at least one curved inlet channel comprises two curved inlet channels.

10. The spin chamber of claim 9, wherein the two curved inlet channels are disposed on opposing sides of the primary recess.

11. The spin chamber of claim 10, wherein the tangential sections of each opposing curved inlet channel are opposite each other across the primary recess, and wherein the funnel sections of each opposing curved inlet channel are opposite each other across the primary recess.

12. The spin chamber of claim 9, wherein a first curved inlet channel of the two curved inlet channels has a greater depth along the longitudinal axis in its tangential section than in its funnel section, and wherein a second curved inlet channel of the two curved inlet channels has a greater depth in its funnel section than in its tangential section.

13. The spin chamber of claim 9, wherein a cross-sectional area of the air inlet of a first of the two curved inlet channels is substantially equal to a cross-sectional area of the air inlet of a second of the two curved inlet channels.

14. The spin chamber of claim 2, wherein the at least one curved inlet channel is configured such that in use air feeds into the primary recess, thereby causing the capsule to be lifted out of the secondary recess and to spin in the primary recess.

15. The spin chamber of claim 2, wherein a bottom surface of the at least one curved inlet channel is substantially level with the bottom surface of the primary recess with respect to the longitudinal axis.

16. The spin chamber of claim 2, wherein the primary recess extends downwards from the top surface of the spin chamber along the longitudinal axis, and the at least one curved inlet channel defines a curved recess extending downwards from the top surface of the spin chamber.

17. An inhaler comprising:
a spin chamber, wherein the spin chamber comprises:
a primary recess configured to receive air to mix with contents of a capsule, the primary recess having a curved wall configured to allow rotation of the capsule;
a secondary recess configured to hold the capsule, the secondary recess located within a bottom surface of the primary recess; and
at least one curved inlet channel configured to allow air travel therethrough, the at least one curved inlet channel defining a curved recess and comprising a tangential section and a funnel section, wherein:
at least a portion of the tangential section is substantially tangential to the curved wall of the primary recess;
the tangential section is connected at a first end to an air inlet on an exterior surface of the spin chamber and at a second end to a first end of the funnel section, wherein the air inlet is configured to allow air to enter therethrough into the spin chamber; and
the funnel section curves in toward the primary recess and is connected at a second end to an entry point configured to allow air enter therethrough into the primary recess, wherein the funnel section is downstream from the tangential section;
wherein the curved inlet channel is separated from the primary recess along a majority of its length by the curved wall of the primary recess.

18. The inhaler of claim 17, wherein the inhaler is configured to allow air to flow in from the air inlet, through the at least one curved inlet channel, through the primary recess and out through an outlet of the inhaler.

19. The inhaler of claim 18, wherein the outlet of the inhaler comprises a mouthpiece.
